(19) United States
(12) Patent Application Publication
(10) Pub. No.: US 2005/0196790 A1
(43) Pub. Date: Sep. 8, 2005

(54) Methods for detection and quantitation of minimum length polymers

(75) Inventor: Jenny E. Rooke, Somerville, MA (US)

Correspondence Address: WOLF GREENFIELD & SACKS, PC;FEDERAL RESERVE PLAZA, 600 ATLANTIC AVENUE, BOSTON, MA, 02210-2211, US

(73) Assignee: U.S. Genomics, Inc., Woburn, MA (US)

(21) Appl. No.: 11/053,738

(22) Filed: Feb. 7, 2005

Related U.S. Application Data
(60) Provisional application No. 60/542,478, filed on Feb. 5, 2004.

(51) Int. Cl. C12Q 1/68

(52) U.S. Cl. 435/6

(57) ABSTRACT

The invention provides methods for detecting and measuring levels of specific polymers that are defined by a minimum length. Such detection methods are applicable to disease detection.

### RELATED APPLICATIONS

This application claims priority to U.S. Provisional Application Ser. No. 60/542,478, entitled “METHODS FOR DETECTION AND QUANTITATION OF MINIMUM LENGTH POLYMERS”, filed Feb. 5, 2004, the entire contents of which are incorporated by reference herein.

### FIELD OF THE INVENTION

The invention relates to the determination of polymers according to size and applications thereof.

### BACKGROUND OF THE INVENTION

Polymers such as deoxyribonucleic acid (DNA) and ribonucleic acid (RNA) are typically present in a wide range of lengths in a cell or biological sample. A fragmented DNA molecule may be as short as a few base pairs, whereas an intact chromosome can comprise millions of contiguous bases. Likewise, RNA molecules produced by a living cell can range from about 21 nucleotides, in the case of a non-coding microRNA (miRNA), to tens of thousands of nucleotides in an intact messenger RNA molecule (mRNA).

Furthermore, variations in the lengths of endogenous DNA and RNA molecules are known to correlate with important functions and processes within the cell. For example, the DNA in a naturally senescing cell is typically degraded into short fragments only a few hundred base pairs long (or less) in a process called apoptosis. In contrast, DNA in cancerous cells can escape programmed cell death degradation to which normal cells are usually subject. As a result, DNA from such cells can exist in relatively long fragments of a kilobase or more. The majority of functional RNA molecules in the cell are processed from longer precursors (e.g., mRNA which are cut from hairpin precursor molecules as well as mature messenger RNA from which intron sequences have been spliced). Thus the length of a specific RNA molecule can indicate the extent to which it has been processed toward its mature form and consequently its ability to perform its natural function within the cell.

In addition to endogenous natural variation, DNA and RNA molecule length can be affected by experimental manipulation such as sample preparation and handling. Restriction enzyme digestion of a DNA sample can be used to cleave longer DNA molecules into smaller fragments. Improper handling of an RNA sample often results in non-specific degradation of the RNA molecules to short fragments, rendering the sample unfit for further manipulation or analysis.

Thus the ability to determine the presence and/or levels of a DNA or RNA molecule of a specific (or minimum) length can yield important information about the biology of the tissue or organism from which the sample was taken and/or about the state of the sample itself. A variety of methods are currently employed in molecular biology to ascertain information about molecular length. Perhaps the simplest method involves the use of gel electrophoresis to achieve size separation of DNA or RNA molecules. Alternatively, the polymerase chain reaction (PCR) binds two sequence-specific oligonucleotide probes to a DNA molecule and subsequently enzymatically amplifies the intervening sequence to produce copies of the template DNA of a specific length. The presence of the specific PCR product (which can be detected by gel electrophoresis, for example) indicates the presence in the original sample of the DNA of corresponding (or minimum) length.

There exists a need for more rapid and less laborious detection, measurement and analysis of polymers such as DNA and RNA.

### SUMMARY OF THE INVENTION

The invention relates in part to methods for detecting polymers of a particular length in a sample. The length of the polymer in some instances is its distinguishing feature and/or the distinguishing or sought-after feature of the sample from which it derived. The invention further provides methods for measuring the levels of such polymers in a sample.

According to one aspect of the invention, a method is provided for determining presence of a target polymer in a sample comprising contacting a sample of one or more polymers with first and second polymer-specific probes labeled with first and second detectable labels respectively, and analyzing the sample for the presence of coincident binding of the first and second polymer-specific probes to a single polymer using a suitable detection system such as a single molecule detection system. The first and second polymer-specific probes bind to unique and distinct target sites separated by a known minimum length within a target polymer. The first and second detectable labels are unique and distinct. Coincident binding of the first and second polymer-specific probes indicates presence of a target polymer in the sample.

In one embodiment, the single molecule detection system is a linear molecule (or polymer) detection system. In important embodiments, the single molecule detection system is a Gene Engine™ system.

In one embodiment, the method further comprises measuring the total number of coincident binding events in a sample as an indicator of the amount of polymer in the sample (i.e., the level of polymer).

In one embodiment, coincident binding of the first and second polymer-specific probes is indicative of a mutant polymer. In another embodiment, coincident binding of the first and second polymer-specific probes is indicative of the presence of an apoptosis-resistant cell. The apoptotic resistant cell may be a cancer cell, such as but not limited to a colorectal cancer cell. In important embodiments, the sample is a bodily sample. In some embodiments, the sample has a nanoliter volume. In other embodiments, the polymer is present at a frequency of 1 in 1,000,000 polymers in the sample.

In one embodiment, the method further comprises contacting the sample with a plurality of polymer-specific probes, wherein each polymer in a sample is detected by a unique pair of probes.

In another embodiment, a plurality of polymers can be detected simultaneously.

In yet another embodiment, the method further comprises comparing coincident binding in two or more samples. In a related embodiment, the two or more samples are harvested at different times. In still another related embodiment, the two or more samples represent normal and diseased tissue.

The polymer may be a nucleic acid but it is not so limited. The nucleic acid may be a DNA or RNA. DNA may be genomic nuclear DNA, genomic mitochondrial DNA or cDNA. RNA may be mRNA or miRNA. In one embodiment, the polymer is not pre-amplified. The polymer and/or the probes may be single stranded. In one embodiment, the polymer is condensed or coiled while in others it is stretched either completely or partially.

In one embodiment, the first and second detectable labels are of the same type. In another embodiment, the first and second detectable labels are independently selected from the group consisting of an electron spin resonance molecule, an electrical charge transferring molecule, a fluorescent molecule, a chemiluminescent molecule, a radioisotope, an enzyme, an enzyme substrate, a chromogenic substrate, a biotin molecule, a streptavidin molecule, a semiconductor nanocrystal, a semiconductor nanoparticle, a colloid gold nanocrystal, a ligand, a microbead, a magnetic bead, a paramagnetic particle, a quantum dot, an affinity molecule, a protein, a peptide, nucleic acid, a carbohydrate, an antigen, a hapten, an antibody, an antibody fragment and a lipid. In one embodiment, the polymer is imaged directly. In an important embodiment, the first and second detectable labels are fluorescent molecules (i.e., fluorophores).

In one embodiment, the polymer is free-flowing. In another embodiment, the polymer is fixed to a solid support. In one embodiment, the polymer is fixed to the solid support in a random orientation. In another embodiment, the polymer is fixed to the solid support in a non-continuous manner (i.e., not every unit of the polymer is fixed to the solid support).

In one embodiment, coincident binding is detected by the direct coincident detection of the first and second detectable labels. The coincident binding may be a color coincident event. In another embodiment, coincident binding is determined by confocal detection.

In another embodiment, coincident binding is proximal binding of the first detectable label that is a donor FRET fluorophore and the second detectable label that is an acceptor FRET fluorophore. Coincident binding may then be detected by a signal from the acceptor FRET fluorophore upon laser excitation of the donor FRET fluorophore.

In one embodiment, the method further comprises performing a column purification step after the contacting step in order to remove unbound probes. In another embodiment, the unbound probes are not removed prior to analysis.

In one embodiment, the probes are comprised of DNA, RNA, PNA, LNA or a combination thereof. In another embodiment, the first and second detectable labels are provided as molecular beacons. In yet another embodiment, at least the first or the second detectable label is attached to a nucleic acid molecule hybridized to a universal linker attached to the first or second polymer-specific unit specific marker.

These and other objects of the invention will be described in further detail herein.

Each of the limitations of the invention can encompass various embodiments of the invention. It is therefore anticipated that each of the limitations of the invention involving any one element or combinations of elements can be included in each aspect of the invention. This invention is not limited in its application to the details of construction and the arrangement of components set forth in the following description or illustrated in the drawings. The invention is capable of other embodiments and of being practiced or of being carried out in various ways.

The phraseology and terminology used herein is for the purpose of description and should not be regarded as limiting. The use of “including”, “comprising”, or “having”, “containing”, “involving”, and variations thereof herein, is meant to encompass the items listed thereafter and equivalents thereof as well as additional items.

### BRIEF DESCRIPTION OF THE DRAWINGS

The accompanying Figures are not intended to be drawn to scale. For purposes of clarity, not every component may be labeled in every Figure.

FIGS. 1A-C are schematics showing coincidence binding and detection of two probes on a target polymer. Coincidence detection on a single polymer can establish the presence of intact polymers of length “n”, where n is the distance between multiple tags specifically bound to the polymer. (A) DNA polymers having probe (or tag) A and probe (or tag) B attached thereto are shown. The distance between the binding site of probe A and probe B is denoted as n bases. (B) A DNA polymer having both probe A and probe B attached thereto is analyzed using a single molecule detection system. The result is overlapping peaks for the probe A signal and the probe B signal, which indicate the presence of a DNA polymer of a minimum length of n bases. (C) A sample of fragmented DNA polymers which bind either probe A or probe B but not both is analyzed by either bulk detection methods or single molecule detection systems. The bulk detection methods simply reveal that an equal amount of probe A signal and probe B signal are detected. No information is achieved regarding the cis or trans relationship of the probes, and the intactness of the polymer. The single molecule detection system however reveals no overlapping peaks for probe A and probe B signals and therefore indicates that no intact molecules are present.

FIGS. 2A and B are schematics showing that coincidence detection can measure intact versus degraded polymers in a sample. (A) Intact RNA polymers having probe A and probe B bound thereto are analyzed resulting in a series of overlapping peaks, each corresponding to an analyzed polymer. (B) Degraded RNA polymers will have a shorter length and thus will be less likely to bind both probes. This results in a single probe being bound to individual polymers and few if any coincidence (or overlapping) peaks.

FIGS. 3A and B are schematics showing the probing and measuring of a plurality of polymers of differing lengths in a sample. Using unique probe pairs, each designed to identify a polymer of a specific minimum length, a plurality of polymers may be analyzed in a given sample and run. Coincident detection of probes A and B denotes a polymer of 1 kb length, while coincident detection of probes A and C denotes a polymer of 2 kb length. The Figure illustrates that one of the probes may recognize and bind to more than one polymer. The method further allows for a relative comparison of the number of polymers of one length versus another.

It is to be understood that the Figures are not required for enablement of the invention.

### DETAILED DESCRIPTION OF THE INVENTION

The invention uses coincident detection of two or more sequence-specific probes placed along a polymer at specific sites (i.e., target sites) to indicate the presence of a specific polymer of minimum length in a sample, where the minimum length is defined by the separation of the target sites along the polymer. Furthermore, the levels of coincident signal detected can be used to quantitate the amount of a specific polymer of minimum length in the sample.

Generally, a population of polymers is exposed to at least two distinguishable and detectable probes for a time and under conditions sufficient to allow the probes to bind to the polymer at specific target sites. The probes are designed to bind to target sites that are separated by a known distance. Accordingly, the minimum length of the polymer is predetermined by the choice of probes and the spacing between their respective target sites. Once the probes and polymer are incubated together for a time and under conditions that would allow for specific binding of the two, each polymer is individually interrogated for a coincident binding event using, for example, a single molecule detection system, such as that described above. The coincident binding event indicates that the two distinguishable and detectable probes are bound to a single polymer, and that the polymer has a minimum length as defined by the distance between the two specific probe sites.

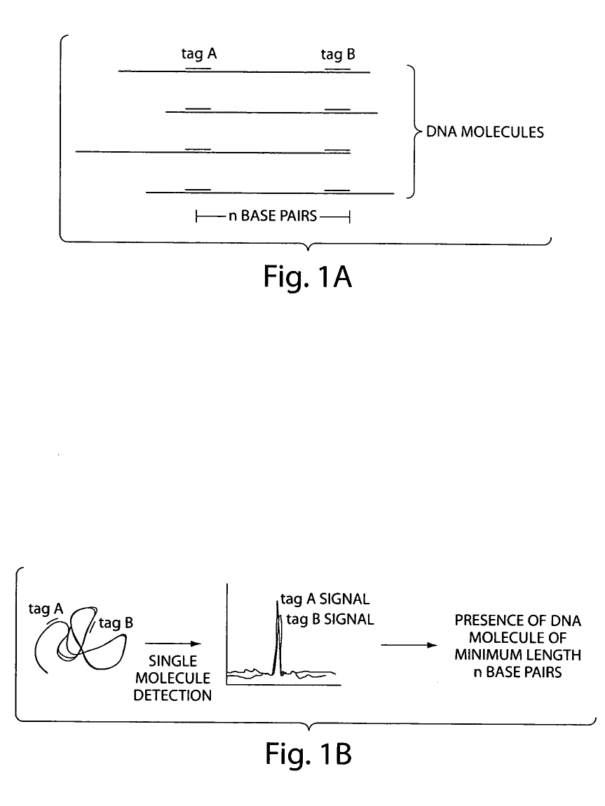

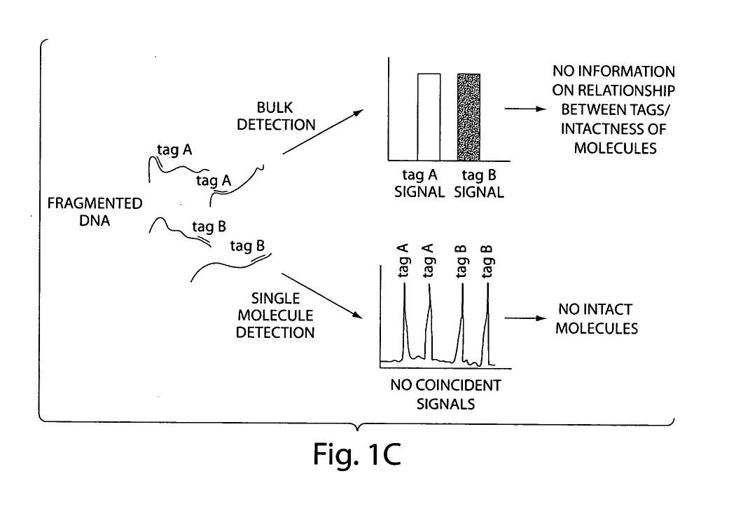

As an example, consider a DNA molecule of potentially variable length contacted with two probes (A and B), each designed to bind to the DNA at a specific and separate site from the other. The distance between the two binding sites is n bases (FIG. 1A). The probes may be oligonucleotides of sufficient length to confer sequence specificity (e.g., a 5-mer or longer), or a DNA-binding protein, or any other agent which binds the DNA at a specific site. The oligonucleotide may be comprised of natural elements or non-naturally occurring elements such as LNAs or PNAs, as discussed in greater detail herein. For detection purposes, the probes may be labeled with any combination of detectable labels as listed herein provided each probe is separately and uniquely detectable. For example, the probes may be labeled with two different fluorescent molecules (i.e., fluorophores). In this example, the coincident detection of both fluorescent molecules on a single polymer is indicative of the presence of the specific DNA molecule of minimum length of n bases (FIG. 1B). At least one probe must be polymer-specific.

Traditional detection technologies rely on bulk fluorescence from a sample and thus would not be able to distinguish between two-color fluorescence bound to a single polymers (as provided by the invention) and two-color fluorescence from probes bound to separate polymers (e.g., some polymers bound to probe A and some polymers bound to probe B, but none to both probe A and B) (FIG. 1C). Using the methods of the invention preferably together with single molecule coincidence analysis, coincident binding of both probes can be assessed, indicating the presence of an intact polymer of minimum length of n bases or base pairs. The method allows such “minimum length” polymers to be distinguished from smaller fragmented polymers that each bind one but not both probes. The actual length of the polymer may be longer than the minimum length set forth by the methods provided herein.

In some instances, particularly where the polymer has been subjected to cleavage and/or shearing, either naturally or experimentally, the polymer may be smaller than the minimum length. In these latter instances, it is expected that preferably one (but sometimes both) probes are no longer capable of binding to the polymer because one or both target sites have been removed. These phenomena can be used in comparison analysis or time course analysis. For example, if a subject is being monitored over a period of time and samples are being harvested from the subject at various time points, then each sample can be analyzed for the presence of coincident events. Fluctuations in coincident events in time may be indicative of biological phenomena occurring with the subject (e.g., senescence of particular cells or of the subject as a whole, efficacy of a particular treatment resulting in normal responses to apoptotic pathways and/or natural telomeric shortening, and the like). Similarly, such comparisons can be made between samples harvested at the same time (e.g., between a suspected diseased tissue and its normal counterpart).

Another application of the information gathered using the methods provided herein is detecting the presence of longer DNA molecules in a sample as an indicator of cells that are not undergoing apoptotic cell death, as described earlier. Maintenance of polymers of at least minimum lengths is indicative that certain cells are not undergoing apoptosis. Resistance to apoptosis is a potential marker for malignant or pre-malignant cells. As stated above, disappearance of polymers of a minimum length may be indicative of the efficacy of a treatment modality directed towards a malignant or pre-malignant condition.

It is to be understood that the method also allows one to determine whether the subject from which the sample came is either at risk of disease or has a disease such as cancer. Examples of cancers include but are not limited to basal cell carcinoma, biliary tract cancer; bladder cancer; bone cancer; brain cancer; breast cancer; cervical cancer; choriocarcinoma; CNS cancer; colon and rectum cancer (i.e., colorectal cancer); connective tissue cancer; cancer of the digestive system; endometrial cancer; esophageal cancer; eye cancer; gastric cancer; cancer of the head and neck; intra-epithelial neoplasm; kidney cancer; larynx cancer; leukemia; acute lymphoid leukemia; acute myeloid leukemia; chronic lymphoid leukemia; chronic myeloid leukemia; liver cancer; lung cancer such as small cell lung cancer and non-small cell lung cancer; lymphoma; Hodgkin's lymphoma; Non-Hodgkin's lymphoma; melanoma; myeloma; neuroblastoma; oral cavity cancer; ovarian cancer; pancreatic cancer; prostate cancer; retinoblastoma; rhabdomyosarcoma; rectal cancer; renal cancer; cancer of the respiratory system; sarcoma; skin cancer; stomach cancer; testicular cancer; thyroid cancer; uterine cancer; and cancer of the urinary system.

In some important embodiments, it is to be understood that the polymer to be detected need not be specific for a particular disease since the method involves determining whether the polymer population as a whole is maintaining its length rather than being degraded due to normal physiological processes that affect all or some polymers. Thus, housekeeping genes expressed by all cells can be the target polymer. In another example, non-expressed DNA or non-translated RNA may be the target polymer.

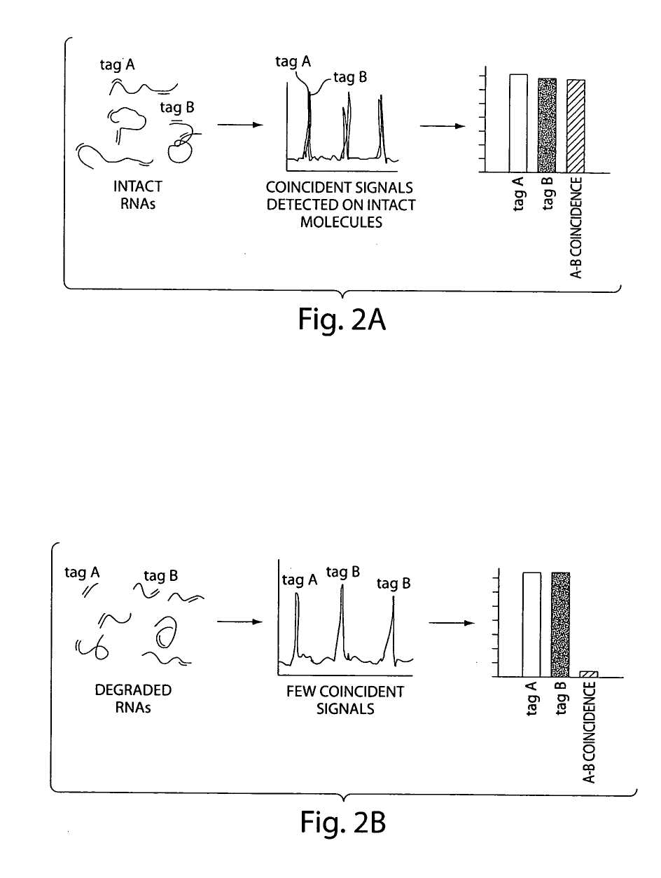

The method provided herein can also be used to quantitate the levels of a polymer of a minimum length. This is accomplished by measuring the number of coincident events since this corresponds to the number of polymers in the sample to which both probes are bound (i.e., which are intact and of minimum length). In this manner, two samples may be analyzed and compared to determine relative levels of a molecule of a certain size. As an example, two RNA samples may be subjected to different sample preparation conditions and then probed with probes specific to the two ends of a specific RNA molecule. The number of coincident events detected in each sample is an indication of the number of intact (rather than degraded) RNA molecules remaining in the sample after sample preparation methods (FIG. 2).

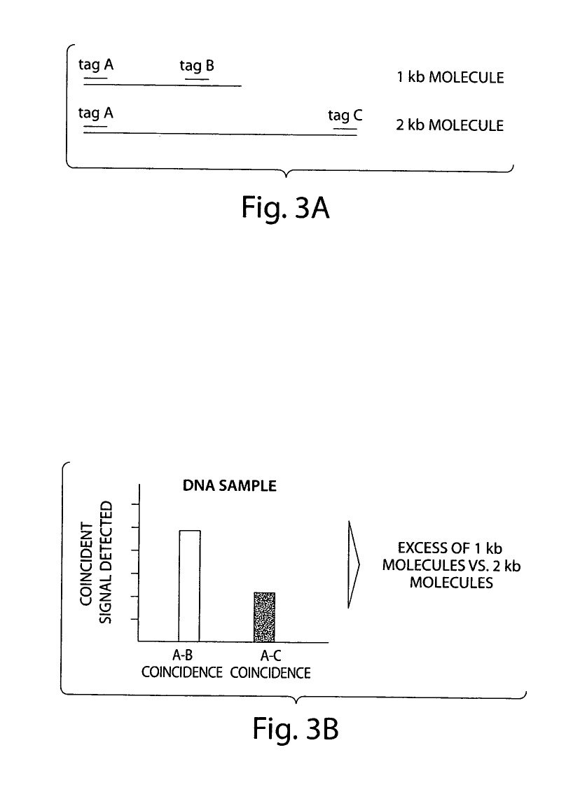

The method can be adapted to introduce additional probes. In this way, a plurality of specific polymers in a sample may be assayed simultaneously to determine their presence as well as their levels (e.g., relative to each other) in a sample (FIG. 3). As used herein, a plurality refers to more than one polymer or probe pair (or more than two probes), and can include at least two, at least three, at least four, at least five, at least six, at least seven, at least eight, at least nine, at least ten or more. In some embodiments, the plurality is one thousand or less, or one hundred or less, or fifty or less.

The method measures coincident detection. As used herein, “coincident detection” refers to detection of two or more probes bound to a single polymer. The probes may be detected simultaneously or sequentially, depending on the application. However, as illustrated in the Figures, coincident detection may be indicated by the presence of overlapping intensity peaks. Probes bound to a single polymer will be analyzed in close temporal proximity to each other than will two probes bound to separate polymers. Similarly, “coincident binding” refers to the binding of two or more probes on a single polymer. Coincident binding of two or more probes is used as an indicator of the polymer of interest, particularly where the sequence of one and preferably both probes is specific and unique to the polymer of interest.

Coincident binding may take many forms including but not limited to a color coincident event, whereby two colors corresponding to the first and second detectable labels are detected. Coincident binding may also be manifest as the proximal binding of a first detectable label that is a donor FRET fluorophore and a second detectable label that is an acceptor FRET fluorophore. In this latter embodiment, a positive signal is a signal from the acceptor FRET fluorophore upon laser excitation of the donor FRET fluorophore. This latter embodiment can be accomplished with a detection and analysis system that comprises only one detector and one laser since a positive signal from the FRET pair is generated by only one laser and is emitted from only one fluorophore.

FRET pairs are two fluorophores that are capable of undergoing FRET to produce or eliminate a detectable signal when positioned in proximity to one another. Examples of donors include Alexa 488, Alexa 546, BODIPY 493, Oyster 556, Fluor (FAM), Cy3 and TTR (Tamra). Examples of acceptors include Cy5, Alexa 594, Alexa 647 and Oyster 656.

A “polymer” as used herein is a compound having a linear backbone to which monomers are linked together by linkages. The polymer is made up of a plurality of individual monomers. An individual monomer as used herein is the smallest building block that can be linked directly or indirectly to other building blocks (or monomers) to form a polymer. At a minimum, the polymer contains at least two linked monomers. The particular type of monomer will depend upon the type of polymer being analyzed. In preferred embodiments, the polymer is a nucleic acid such as a DNA or RNA molecule. The invention however is not so limited and could be used to label and analyze non-nucleic acid polymers. The polymer may be a protein, a peptide, an oligo- or polysaccharide, a lipid, etc. The polymer must however be capable of being bound to or by sequence- or structure-specific probes, wherein the sequence or structure recognized and bound by the probe is unique to that polymer or to a region of the polymer. It is possible to use a given probe for two or more polymers, provided that the probe pair is still specific for only a given polymer.

The polymers to be detected are referred to herein as “target” polymers. In some important embodiments, the target polymers are DNA, RNA, or amplification products or intermediates thereof, including complementary DNA (cDNA). In important embodiments, the nucleic acid molecules are DNA. DNA includes genomic DNA (such as nuclear DNA and mitochondrial DNA), as well as in some instances cDNA.

The samples to be tested for polymer presence can be biological or bodily samples such as tissue biopsies, urine, sputum, semen, stool, saliva and the like. The sample in some instances can be analyzed as is without harvest and isolation of polymers contained therein. Alternatively, harvest and isolation of nucleic acid molecules can be performed and methods for doing so are routinely practiced in the art and can be found in standard molecular biology textbooks (e.g., such as Maniatis' Handbook of Molecular Biology).

Preferably, prior amplification using techniques such as polymerase chain reaction (PCR) are not necessary. Accordingly, the polymer may be a non in vitro amplified nucleic acid. As used herein, a “non in vitro amplified nucleic acid” refers to a nucleic acid that has not been amplified in vitro using techniques such as polymerase chain reaction or recombinant DNA methods. A non in vitro amplified nucleic acid may however be a nucleic acid that is amplified in vivo (in the biological sample from which it was harvested) as a natural consequence of the development of the cells in vivo. This means that the non in vitro nucleic acid may be one which is amplified in vivo as part of locus amplification, which is commonly observed in some cell types as a result of mutation or cancer development.

In important embodiments, the sample has a nanoliter volume. That is, it is only necessary to load a nanoliter volume into the detection system in order to perform the method described herein. In still other important embodiments, the polymer is present at a frequency of 1 in 1,000,000 polymers or 1 in 2,000,000 polymers in the sample. Accordingly, the method can be used to detect and analyze polymers that are extremely rare. It is to be understood that in some embodiments, all the manipulations described herein may be accomplished within the detection system, thereby reducing any sample loss.

The term “nucleic acid” refers to multiple linked nucleotides (i.e., molecules comprising a sugar (e.g., ribose or deoxyribose) linked to an exchangeable organic base, which is either a pyrimidine (e.g., cytosine (C), thymidine (T) or uracil (U)) or a purine (e.g., adenine (A) or guanine (G)). “Nucleic acid” and “nucleic acid molecule” are used interchangeably and refer to oligoribonucleotides as well as oligodeoxyribonucleotides. The terms shall also include polynucleosides (i.e., a polynucleotide minus a phosphate) and any other organic base containing nucleic acid. The organic bases include adenine, uracil, guanine, thymine, cytosine and inosine. Nucleic acids can be obtained from natural sources, or can be synthesized using a nucleic acid synthesizer.

As used herein with respect to linked units of a polymer including a nucleic acid, “linked” or “linkage” means two entities bound to one another by any physicochemical means. Any linkage known to those of ordinary skill in the art, covalent or non-covalent, is embraced. Natural linkages, which are those ordinarily found in nature connecting for example the individual units of a particular nucleic acid, are most common. Natural linkages include, for instance, amide, ester and thioester linkages. The individual units of a nucleic acid analyzed by the methods of the invention may be linked, however, by synthetic or modified linkages. Nucleic acids where the units are linked by covalent bonds will be most common but those that include hydrogen bonded units are also embraced by the invention. It is to be understood that all possibilities regarding nucleic acids apply equally to nucleic acid targets and nucleic acid probes.

The nucleic acids may be double-stranded, although in some embodiments the nucleic acid targets are denatured and presented in a single-stranded form. This can be accomplished by modulating the environment of a double-stranded nucleic acid including singly or in combination increasing temperature, decreasing salt concentration, and the like. Methods of denaturing nucleic acids are known in the art.

In some embodiments, the invention embraces nucleic acid derivatives as polymers and/or probes. As used herein, a “nucleic acid derivative” is a non-naturally occurring nucleic acid or a unit thereof. Nucleic acid derivatives may contain non-naturally occurring elements such as non-naturally occurring nucleotides and non-naturally occurring backbone linkages. These include substituted purines and pyrimidines such as C-5 propyne modified bases, 5-methylcytosine, 2-aminopurine, 2-amino-6-chloropurine, 2,6-diaminopurine, hypoxanthine, 2-thiouracil and pseudoisocytosine. Other such modifications are well known to those of skill in the art.

The nucleic acid derivatives may also encompass substitutions or modifications, such as in the bases and/or sugars. For example, they include nucleic acids having backbone sugars which are covalently attached to low molecular weight organic groups other than a hydroxyl group at the 3′ position and other than a phosphate group at the 5′ position. Thus, modified nucleic acids may include a 2′-O-alkylated ribose group. In addition, modified nucleic acids may include sugars such as arabinose instead of ribose.

The nucleic acids may be heterogeneous in backbone composition thereby containing any possible combination of nucleic acid units linked together such as peptide nucleic acids (which have amino acid linkages with nucleic acid bases, and which are discussed in greater detail herein). In some embodiments, the nucleic acids are homogeneous in backbone composition.

The target nucleic acids commonly have a phosphodiester backbone because this backbone is most common in vivo. However, they are not so limited. Backbone modifications are known in the art. One of ordinary skill in the art is capable of preparing such nucleic acids without undue experimentation. The probes, if nucleic acid in nature, can also have backbone modifications such as those described above.

The polymers and probes if comprising nucleic acid components can be stabilized in part by the use of backbone modifications. The invention intends to embrace, in addition to the peptide and locked nucleic acids discussed herein, the use of the other backbone modifications such as but not limited to phosphorothioate linkages, phosphodiester modified nucleic acids, combinations of phosphodiester and phosphorothioate nucleic acid, methylphosphonate, alkylphosphonates, phosphate esters, alkylphosphonothioates, phosphoramidates, carbamates, carbonates, phosphate triesters, acetamidates, carboxymethyl esters, methylphosphorothioate, phosphorodithioate, p-ethoxy, and combinations thereof.

In some embodiments, the polymer or probe is a nucleic acid that is a peptide nucleic acid (PNA), a bisPNA clamp, a pseudocomplementary PNA, a locked nucleic acid (LNA), DNA, RNA, or co-nucleic acids of the above such as DNA-LNA co-nucleic acids. siRNA or miRNA or RNAi molecules can be similarly used. In some instances, the nucleic acid target can also be comprised of any of these elements.

In some embodiments, the probe is a peptide nucleic acid (PNA), a bisPNA clamp, a locked nucleic acid (LNA), a ssPNA, a pseudocomplementary PNA (pcPNA), a two-armed PNA (as described in co-pending U.S. patent application having Ser. No. 10/421,644 and publication number U.S. 2003-0215864 A1 and published Nov. 20, 2003, and PCT application having serial number PCT/US03/12480 and publication number WO 03/091455 A1 and published Nov. 6, 2003, filed on Apr. 23, 2003), or co-polymers thereof (e.g., a DNA-LNA co-polymer).

PNAs are DNA analogs having their phosphate backbone replaced with 2-aminoethyl glycine residues linked to nucleotide bases through glycine amino nitrogen and methylenecarbonyl linkers. PNAs can bind to both DNA and RNA targets by Watson-Crick base pairing, and in so doing form stronger hybrids than would be possible with DNA or RNA based probes.

PNAs are synthesized from monomers connected by a peptide bond (Nielsen, P. E. et al. Peptide Nucleic Acids, Protocols and Applications, Norfolk: Horizon Scientific Press, p. 1-19 (1999)). They can be built with standard solid phase peptide synthesis technology. PNA chemistry and synthesis allows for inclusion of amino acids and polypeptide sequences in the PNA design. For example, lysine residues can be used to introduce positive charges in the PNA backbone. All chemical approaches available for the modifications of amino acid side chains are directly applicable to PNAs.

PNA has a charge-neutral backbone, and this attribute leads to fast hybridization rates of PNA to DNA (Nielsen, P. E. et al. Peptide Nucleic Acids, Protocols and Applications, Norfolk: Horizon Scientific Press, p. 1-19 (1999)). The hybridization rate can be further increased by introducing positive charges in the PNA structure, such as in the PNA backbone or by addition of amino acids with positively charged side chains (e.g., lysines). PNA can form a stable hybrid with DNA molecule. The stability of such a hybrid is essentially independent of the ionic strength of its environment (Orum, H. et al., BioTechniques 19(3):472-480 (1995)), most probably due to the uncharged nature of PNAs. This provides PNAs with the versatility of being used in vivo or in vitro. However, the rate of hybridization of PNAs that include positive charges is dependent on ionic strength, and thus is lower in the presence of salt.

Several types of PNA designs exist, and these include single strand PNA (ssPNA), bisPNA and pseudocomplementary PNA (pcPNA).

The structure of PNA/DNA complex depends on the particular PNA and its sequence. Single stranded PNA (ssPNA) binds to single stranded DNA (ssDNA) preferably in antiparallel orientation (i.e., with the N-terminus of the ssPNA aligned with the 3′ terminus of the ssDNA) and with a Watson-Crick pairing. PNA also can bind to DNA with a Hoogsteen base pairing, and thereby forms triplexes with double stranded DNA (dsDNA) (Wittung, P. et al., Biochemistry 36:7973 (1997)).

Single strand PNA is the simplest of the PNA molecules. This PNA form interacts with nucleic acids to form a hybrid duplex via Watson-Crick base pairing. The duplex has different spatial structure and higher stability than dsDNA (Nielsen, P. E. et al. Peptide Nucleic Acids, Protocols and Applications, Norfolk: Horizon Scientific Press, p. 1-19 (1999)). However, when different concentration ratios are used and/or in presence of complimentary DNA strand, PNA/DNA/PNA or PNA/DNA/DNA triplexes can also be formed (Wittung, P. et al., Biochemistry 36:7973 (1997)). The formation of duplexes or triplexes additionally depends upon the sequence of the PNA. Thymine-rich homopyrimidine ssPNA forms PNA/DNA/PNA triplexes with dsDNA targets where one PNA strand is involved in Watson-Crick antiparallel pairing and the other is involved in parallel Hoogsteen pairing. Cytosine-rich homopyrimidine ssPNA preferably binds through Hoogsteen pairing to dsDNA forming a PNA/DNA/DNA triplex. If the ssPNA sequence is mixed, it invades the dsDNA target, displaces the DNA strand, and forms a Watson-Crick duplex. Polypurine ssPNA also forms triplex PNA/DNA/PNA with reversed Hoogsteen pairing.

BisPNA includes two strands connected with a flexible linker. One strand is designed to hybridize with DNA by a classic Watson-Crick pairing, and the second is designed to hybridize with a Hoogsteen pairing. The target sequence can be short (e.g., 8 bp), but the bisPNA/DNA complex is still stable as it forms a hybrid with twice as many (e.g., a 16 bp) base pairings overall. The bisPNA structure further increases specificity of their binding. As an example, binding to an 8 bp site with a probe having a single base mismatch results in a total of 14 bp rather than 16 bp.

Preferably, bisPNAs have homopyrimidine sequences, and even more preferably, cytosines are protonated to form a Hoogsteen pair to a guanosine. Therefore, bisPNA with thymines and cytosines is capable of hybridization to DNA only at pH below 6.5. The first restriction—homopyrimidine sequence only—is inherent to the mode of bisPNA binding. Pseudoisocytosine (J) can be used in the Hoogsteen strand instead of cytosine to allow its hybridization through a broad pH range (Kuhn, H., J. Mol. Biol. 286:1337-1345 1999)).

BisPNAs have multiple modes of binding to nucleic acids (Hansen, G. I. et al., J. Mol. Biol. 307(1):67-74 (2001)). One isomer includes two bisPNA molecules instead of one. It is formed at higher bisPNA concentration and has a tendency to rearrange into the complex with a single bisPNA molecule. Other isomers differ in positioning of the linker around the target DNA strands. All the identified isomers still bind to the same binding site/target.

Pseudocomplementary PNA (pcPNA) (Izvolsky, K. I. et al., Biochemistry 10908-10913 (2000)) involves two single stranded PNAs added to dsDNA. One pcPNA strand is complementary to the target sequence, while the other is complementary to the displaced DNA strand. As the PNA/DNA duplex is more stable, the displaced DNA generally does not restore the dsDNA structure. The PNA/PNA duplex is more stable than the DNA/PNA duplex and the PNA components are self-complementary because they are designed against complementary DNA sequences. Hence, the added PNAs would rather hybridize to each other. To prevent the self-hybridization of pcPNA units, modified bases are used for their synthesis including 2,6-diamiopurine (D) instead of adenine and 2-thiouracil (^{S}U) instead of thymine. While D and ^{S}U are still capable of hybridization with T and A respectively, their self-hybridization is sterically prohibited.

Locked nucleic acid (LNA) molecules form hybrids with DNA, which are at least as stable as PNA/DNA hybrids (Braasch, D. A. et al., Chem & Biol. 8(1):1-7(2001)). Therefore, LNA can be used just as PNA molecules would be. LNA binding efficiency can be increased in some embodiments by adding positive charges to it. LNAs have been reported to have increased binding affinity inherently.

Commercial nucleic acid synthesizers and standard phosphoramidite chemistry are used to make LNAs. Therefore, production of mixed LNA/DNA sequences is as simple as that of mixed PNA/peptide sequences. The stabilization effect of LNA monomers is not an additive effect. The monomer influences conformation of sugar rings of neighboring deoxynucleotides shifting them to more stable configurations (Nielsen, P. E. et al. Peptide Nucleic Acids, Protocols and Applications, Norfolk: Horizon Scientific Press, p. 1-19 (1999)). Also, lesser number of LNA residues in the sequence dramatically improves accuracy of the synthesis. Naturally, most of biochemical approaches for nucleic acid conjugations are applicable to LNA/DNA constructs.

Other backbone modifications, particularly those relating to PNAs, include peptide and amino acid variations and modifications. Thus, the backbone constituents of PNAs may be peptide linkages, or alternatively, they may be non-peptide linkages. Examples include acetyl caps, amino spacers such as 8-amino-3,6-dioxaoctanoic acid (referred to herein as O-linkers), amino acids such as lysine (particularly useful if positive charges are desired in the PNA), and the like. Various PNA modifications are known and probes incorporating such modifications are commercially available from sources such as Boston Probes, Inc.

The methods of the invention analyze polymers using probes that recognize and bind to specific sites within a polymer. The specific site is preferably a particular linear arrangement of monomers (i.e., a particular defined sequence of monomers) within a target polymer. For example, a target site in a nucleic acid consists of a particular sequence of nucleotides linked to one another. The site may be of any length.

The probe may itself be a polymer but it is not so limited. Examples of suitable probes are nucleic acids (e.g., as probes for target nucleic acids) and peptides and polypeptides (e.g., as probes for target polymers that are nucleic acids or peptides or proteins). As used herein a “peptide” is a polymer of amino acids connected preferably but not solely with peptide bonds. Other probes include but are not limited to sequence-specific major and minor groove binders and intercalators, nucleic acid binding peptides or proteins, sequence-specific peptide-nucleic acids (PNAs), and peptide binding proteins, etc. Preferably, probes are nucleic acids.

With the advent of aptamer technology, it is possible to use nucleic acid based probes (i.e., unit specific markers) in order to recognize and bind a variety of compounds, including peptides and carbohydrates, in a structurally, and thus sequence, specific manner.

The methods provided herein involve the use of probes that bind to the target polymer in a sequence-specific manner. “Sequence-specific” when used in the context of a nucleic acid means that the probe recognizes a particular linear (or in some instances quasi-linear) arrangement of nucleotides or derivatives thereof. In some embodiments, the probes are “polymer-specific” meaning that they bind specifically to a sequence or structure in a polymer. “Specific binding” means the probe binds with greater affinity to the target polymer or a region within the target polymer than it does to other polymers or other polymer regions. It should be possible to achieve conditions under which the probe binds only to the particular target polymer (to which it was designed) and no other polymer. Such “stringent hybridization conditions” are known in the art. (See for example Maniatis' Handbook of Molecular Biology.).

Any molecule that is capable of recognizing a polymer such as a nucleic acid with structural or sequence specificity can be used as a sequence-specific probe. In most instances, such probes will form at least a Watson-Crick bond with the nucleic acid polymer. In other instances, the nucleic acid probe can form a Hoogsteen bond with the nucleic acid polymer, thereby forming a triplex. A nucleic acid probe that binds by Hoogsteen binding enters the major groove of a nucleic acid polymer and hybridizes with the bases located there. Examples of these latter probes include molecules that recognize and bind to the minor and major grooves of nucleic acids (e.g., some forms of antibiotics). In some embodiments, the nucleic acid probes can form both Watson-Crick and Hoogsteen bonds with the nucleic acid polymer. BisPNA probes, for instance, are capable of both Watson-Crick and Hoogsteen binding to a nucleic acid.

The length of probe can also determine the specificity of binding. The energetic cost of a single mismatch between the probe and the nucleic acid polymer is relatively higher for shorter sequences than for longer ones. Therefore, hybridization of smaller nucleic acid probes is more specific than is hybridization of longer nucleic acid probes because the longer probes can embrace mismatches and still continue to bind to the polymer depending on the conditions. One potential limitation to the use of shorter probes however is their inherently lower stability at a given temperature and salt concentration. In order to avoid this latter limitation, bisPNA probes can be used to bind shorter sequences with sufficient hybrid stability. Longer probes are desirable when unique gene-specific sequences are being detected.

Notwithstanding these provisos, the nucleic acid probes of the invention can be any length ranging from at least 4 nucleotides to in excess of 1000 nucleotides. In preferred embodiments, the probes are 5-100 nucleotides in length, more preferably between 5-25 nucleotides in length, and even more preferably 5-12 nucleotides in length. The length of the probe can be any length of nucleotides between and including the ranges listed herein, as if each and every length was explicitly recited herein. Thus, the length may be at least 5 nucleotides, at least 10 nucleotides, at least 15 nucleotides, at least 20 nucleotides, or at least 25 nucleotides, or more, in length. The length may range from at least 4, at least 5, at least 6, at least 7, at least 8, at least 9, at least 10, at least 12, at least 15, at least 20, at least 25, at least 50, at least 75, at least 100, at least 150, at least 200, at least 250, at least 500, or more nucleotides (including every integer therebetween as if explicitly recited herein).

It should be understood that not all residues of the probe need hybridize to complementary residues in the nucleic acid target. For example, the probe may be 50 residues in length, yet only 25 of those residues hybridize to the nucleic acid target. Preferably, the residues that hybridize are contiguous with each other.

The probes are preferably single-stranded, but they are not so limited. For example, when the probe is a bisPNA it can adopt a secondary structure with the nucleic acid polymer resulting in a triple helix conformation, with one region of the bisPNA clamp forming Hoogsteen bonds with the backbone of the polymer and another region of the bisPNA clamp forming Watson-Crick bonds with the nucleotide bases of the polymer.

The nucleic acid probe hybridizes to a complementary sequence within the nucleic acid polymer. The specificity of binding can be manipulated based on the hybridization conditions. For example, salt concentration and temperature can be modulated in order to vary the range of sequences recognized by the nucleic acid probes. Those of ordinary skill in the art will be able to determine optimum conditions for a desired specificity.

In some embodiments, the probes may be molecular beacons. When unbound to their targets, the molecular beacon probes form a hairpin structure and do not emit fluorescence since one end of the molecular beacon is a quencher molecule. However, once bound to their targets, the fluorescent and quenching ends of the probe are sufficiently separated so that the fluorescent end can now emit.

In some embodiments, the method can be used to detect a plurality of different polymers in a single analysis. In these cases, the sample is contacted with a plurality of probe pairs, each pair being specific for a particular polymer. Alternatively, some probes may bind to more than one polymer provided that the other probe is specific and unique for the target polymer.

Binding of a probe to a nucleic acid indicates the presence and location of a target site in the target nucleic acid. As used herein, a polymer that is bound by a probe is “labeled” with the probe and/or its detectable label.

Although the single nucleic acid may be linearized or stretched prior to analysis, this is not necessary if the detection system is capable of analyzing both stretched and condensed nucleic acids. This is particularly the case when coincident events are being detected since these events simply require the presence or absence of at least two labels, but are not necessarily dependent upon the relative positioning of the labels (provided they are sufficiently proximal to each other, in some instances, to enable energy transfer from one label to another, for example, where FRET is used).

As used herein, stretching of the target polymer means that the polymer is provided in a substantially linear extended form rather than a compacted, coiled and/or folded form. Stretching the polymer prior to analysis may be accomplished using particular configurations of, for example, a single molecule detection system, in order to maintain the linear form. These configurations are not required if the target polymer can be analyzed in a compacted form.

The sample may be cleaned prior to analyzing the polymer. As used herein “cleaning” refers to the process of removing unbound probes and/or non-hybridized polymers. This cleaning step can be accomplished in a number of ways including but not limited to column purification. Column purification generally involves capture of small molecules within a column with flow-through of larger molecules (such as the target polymers). It is to be understood however that the method can be performed without removal of these reagents prior to analysis, particularly since coincident detection can distinguish between desired hybridization events and artifacts. Thus, in some embodiments, the unbound detectable labels are not removed prior to analysis using the single molecule detection system.

Detectable labels may be, for example, light emitting, energy accepting, fluorescing, radioactive, quenching, and the like, as the invention is not limited in this respect. Guidelines for selecting the appropriate labels, and methods for adding extrinsic labels to polymers are provided in more detail in U.S. Pat. No. 6,355,420 B1.

The detectable label can be directly or indirectly detected. A directly detectable moiety is one that can be detected directly by its ability to emit and/or absorb light of a particular wavelength. An indirectly detectable moiety is one that can be detected indirectly by its ability to bind, recruit and, in some cases, cleave another moiety which itself may emit or absorb light of a particular wavelength. An example of indirect detection is the use of a first enzyme label which cleaves a substrate into directly detectable products. The label may be organic or inorganic in nature. For example, it may be chemical, peptide or nucleic acid in nature although it is not so limited. Labels can be conjugated to a polymer or probe using thiol, amino or carboxylic groups.

The labels described herein are referred to according to the systems by which they are detected. As an example, a fluorescent molecule is a molecule that can be detected using a system of detection that relies on fluorescence (i.e., a fluorescence detection system).

The label may be selected from the group consisting of an electron spin resonance molecule (such as for example nitroxyl radicals), a fluorescent molecule (i.e., fluorophores), a chemiluminescent molecule (e.g., chemiluminescent substrates), a radioisotope, an optical or electron density marker, an enzyme, an enzyme substrate, a biotin molecule, a streptavidin molecule, an electrical charge transferring molecule (i.e., an electrical charge transducing molecule), a chromogenic substrate, a semiconductor nanocrystal, a semiconductor nanoparticle, a colloid gold nanocrystal, a ligand, a microbead, a magnetic bead, a paramagnetic particle, a quantum dot, an affinity molecule, a protein, a peptide, nucleic acid, a carbohydrate, an antigen, a hapten, an antibody, an antibody fragment, and a lipid. They are not so limited however.

More specifically, the detectable label may be selected from the group consisting of directly detectable labels such as a fluorescent molecule (e.g., fluorescein, rhodamine, tetramethylrhodamine, R-phycoerythrin, Cy-3, Cy-5, Cy-7, Texas Red, Phar-Red, allophycocyanin (APC), fluorescein amine, eosin, dansyl, umbelliferone, 5-carboxyfluorescein (FAM), 2′7′-dimethoxy-4′5′-dichloro-6-carboxyfluorescein (JOE), 6 carboxyrhodamine (R6G), N,N,N′,N′-tetramethyl-6-carboxyrhodamine (TAMRA), 6-carboxy-X-rhodamine (ROX), 4-(4′-dimethylaminophenylazo)benzoic acid (DABCYL), 5-(2′-aminoethyl)aminonaphthalene-1-sulfonic acid (EDANS), 4-acetamido-4′-isothiocyanatostilbene-2,2′disulfonic acid, acridine, acridine isothiocyanate, r-amino-N-(3-vinylsulfonyl)phenylnaphthalimide-3,5, disulfonate (Lucifer Yellow VS), N-(4-anilino-1-naphthyl)maleimide, anthranilamide, Brilliant Yellow, coumarin, 7-amino-4-methylcoumarin, 7-amino-4-trifluoromethylcouluarin (Coumarin 151), cyanosine, 4′, 6-diaminidino-2-phenylindole (DAPI), 5′,5″-diaminidino-2-phenylindole (DAPI), 5′,5″-dibromopyrogallol-sulfonephthalein (Bromopyrogallol Red), 7-diethylamino-3-(4′-isothiocyanatophenyl)-4-methylcoumarin diethylenetriamine pentaacetate, 4,4′-diisothiocyanatodihydro-stilbene-2,2′-disulfonic acid, 4,4′-diisothiocyanatostilbene-2,2′-disulfonic acid, 4-dimethylaminophenylazophenyl-4′-isothiocyanate (DABITC), eosin isothiocyanate, erythrosin B, erythrosin isothiocyanate, ethidium, 5-(4,6-dichlorotriazin-2-yl) aminofluorescein (DTAF), QFITC (XRITC), fluorescamine, IR144, IR1446, Malachite Green isothiocyanate, 4-methylumbelliferone, ortho cresolphthalein, nitrotyrosine, pararosaniline, Phenol Red, B-phycoerythrin, o-phthaldialdehyde, pyrene, pyrene butyrate, succinimidyl 1-pyrene butyrate, Reactive Red 4 (Cibacron. RTM. Brilliant Red 3B-A), lissamine rhodamine B sulfonyl chloride, rhodamine B, rhodamine 123, rhodamine X, sulforhodamine B, sulforhodamine 101, sulfonyl chloride derivative of sulforhodamine 101, tetramethyl rhodamine, riboflavin, rosolic acid, and terbium chelate derivatives), a chemiluminescent molecule, a bioluminescent molecule, a chromogenic molecule, a radioisotope (e.g., P³² or H³, ¹⁴C, ¹²⁵I and ¹³¹I), an electron spin resonance molecule (such as for example nitroxyl radicals), an optical or electron density molecule, an electrical charge transducing or transferring molecule, an electromagnetic molecule such as a magnetic or paramagnetic bead or particle, a semiconductor nanocrystal or nanoparticle (such as quantum dots described for example in U.S. Pat. No. 6,207,392 and commercially available from Quantum Dot Corporation and Evident Technologies), a colloidal metal, a colloid gold nanocrystal, a nuclear magnetic resonance molecule, and the like.

The detectable label can also be selected from the group consisting of indirectly detectable labels such as an enzyme (e.g., alkaline phosphatase, horseradish peroxidase, β-galactosidase, glucoamylase, lysozyme, luciferases such as firefly luciferase and bacterial luciferase (U.S. Pat. No. 4,737,456); saccharide oxidases such as glucose oxidase, galactose oxidase, and glucose-6-phosphate dehydrogenase; heterocyclic oxidases such as uricase and xanthine oxidase coupled to an enzyme that uses hydrogen peroxide to oxidize a dye precursor such as HRP, lactoperoxidase, or microperoxidase), an enzyme substrate, an affinity molecule, a ligand, a receptor, a biotin molecule, an avidin molecule, a streptavidin molecule, an antigen (e.g., epitope tags such as the FLAG or HA epitope), a hapten (e.g., biotin, pyridoxal, digoxigenin fluorescein and dinitrophenol), an antibody, an antibody fragment, a microbead, and the like.

Antibodies can be used according to the invention as labels as well as probes. Thus, polymers can be labeled using antibodies or antibody fragments or optionally their corresponding antigens, haptens or epitopes. In the latter embodiment, the antigen, hapten, or epitope may itself be labeled. Detection of bound antibodies is accomplished by techniques known to those skilled in the art. Antibodies bound to polymers can be detected by linking a label to the antibodies and then observing the site of the label. If antibody binding indicates sequence information, then the antibody should bind to the polymer in a sequence-specific manner. If antibody binding indicates merely the presence of the polymer (e.g., represents the backbone of the polymer, as discussed below), then the antibody need not bind to the polymer in a sequence-specific manner. In addition to the use of antigens, haptens and epitopes, antibodies can also be visualized using secondary antibodies or fragments thereof that are specific for the primary antibody. Polyclonal and monoclonal antibodies may be used. Antibody fragments include Fab, F(ab)₂, Fd and antibody fragments which include an antigen binding domain.

Thus, in some embodiments, the polymer is capable of being imaged directly (i.e., it has bound to it via a probe a directly detectable label such as a fluorophore or a radioisotope). In other embodiments, the polymer is imaged indirectly (i.e., it has bound to it via a probe a label that is indirectly detectable (i.e., an enzyme that converts a substrate into a visible product, or a biotin molecule that is bound by a directly labeled avidin molecule, or a primary antibody that is recognized by a secondary antibody or a hapten that is itself directly labeled).

In some embodiments, the polymer and/or probes are labeled with detectable moieties that emit distinguishable signals that can all be detected using one type of detection system. For example, the detectable moieties can all be fluorescent labels or they can all be radioactive labels. In other embodiments, the polymers and/or probes are labeled with moieties that are detected using different detection systems. For example, one polymer or probe may be labeled with a fluorophore while another may be labeled with a radioisotope.

In some instances, it may be desirable to further label the polymer with a standard marker. The standard marker may be used to identify the polymer including defining its ends. For example, the standard marker may be a backbone label. One subset of backbone labels for nucleic acids are nucleic acid stains that bind nucleic acids in a non-sequence-specific manner. Examples include intercalating dyes such as phenanthridines and acridines (e.g., ethidium bromide, propidium iodide, hexidium iodide, dihydroethidium, ethidium homodimer-1 and -2, ethidium monoazide, and ACMA); minor grove binders such as indoles and imidazoles (e.g., Hoechst 33258, Hoechst 33342, Hoechst 34580 and DAPI); and miscellaneous nucleic acid stains such as acridine orange (also capable of intercalating), 7-AAD, actinomycin D, LDS751, and hydroxystilbamidine. All of the aforementioned nucleic acid stains are commercially available from suppliers such as Molecular Probes, Inc.

Still other examples of nucleic acid stains include the following dyes from Molecular Probes: cyanine dyes such as SYTOX Blue, SYTOX Green, SYTOX Orange, POPO-1, POPO-3, YOYO-1, YOYO-3, TOTO-1, TOTO-3, JOJO-1, LOLO-1, BOBO-1, BOBO-3, PO-PRO-1, PO-PRO-3, BO-PRO-1, BO-PRO-3, TO-PRO-1, TO-PRO-3, TO-PRO-5, JO-PRO-1, LO-PRO-1, YO-PRO-1, YO-PRO-3, PicoGreen, OliGreen, RiboGreen, SYBR Gold, SYBR Green I, SYBR Green II, SYBR DX, SYTO-40, -41, -42, -43, -44, -45 (blue), SYTO-13, -16, -24, -21, -23, -12, -11, -20, -22, -15, -14, -25 (green), SYTO-81, -80, -82, -83, -84, -85 (orange), SYTO-64, -17, -59, -61, -62, -60, -63 (red).

In some instances, the detectable labels are part of a FRET system with fluorescence signals dependent upon the proximal location of donor and acceptor molecules.

As used herein, “conjugated” means two entities stably bound to one another by any physicochemical means. It is important that the nature of the attachment is such that it does not substantially impair the effectiveness of either entity. Keeping these parameters in mind, any covalent or non-covalent linkage known to those of ordinary skill in the art is contemplated unless explicitly stated otherwise herein. Non-covalent conjugation includes hydrophobic interactions, ionic interactions, high affinity interactions such as biotin-avidin and biotin-streptavidin complexation and other affinity interactions. Such means and methods of attachment are known to those of ordinary skill in the art. Conjugation can be performed using standard techniques common to those of ordinary skill in the art. For example, U.S. Pat. Nos. 3,940,475 and 3,645,090 demonstrate conjugation of fluorophores and enzymes to antibodies.

The various components described herein can be conjugated to each other by any mechanism known in the art. For instance, functional groups which are reactive with various labels include, but are not limited to, (functional group: reactive group of light emissive compound) activated ester:amines or anilines; acyl azide:amines or anilines; acyl halide:amines, anilines, alcohols or phenols; acyl nitrile:alcohols or phenols; aldehyde:amines or anilines; alkyl halide:amines, anilines, alcohols, phenols or thiols; alkyl sulfonate:thiols, alcohols or phenols; anhydride:alcohols, phenols, amines or anilines; aryl halide:thiols; aziridine:thiols or thioethers; carboxylic acid:amines, anilines, alcohols or alkyl halides; diazoalkane:carboxylic acids; epoxide:thiols; haloacetamide:thiols; halotriazine:amines, anilines or phenols; hydrazine:aldehydes or ketones; hydroxyamine:aldehydes or ketones; imido ester:amines or anilines; isocyanate:amines or anilines; and isothiocyanate:amines or anilines.

The nature of the detection system used will depend upon the nature of the detectable moiety attached to the polymer. The detection system can be selected from any number of detection systems known in the art. These include an electron spin resonance (ESR) detection system, a charge coupled device (CCD) detection system, an avalanche photodiode (APD) detection system, a photomultiplier (PMT) detection system, a fluorescent detection system, an electrical detection system, a photographic film detection system, a chemiluminescent detection system, an enzyme detection system, an atomic force microscopy (AFM) detection system, a scanning tunneling microscopy (STM) detection system, an optical detection system, a nuclear magnetic resonance (NMR) detection system, a near field detection system, and a total internal reflection (TIR) detection system, many of which are electromagnetic detection systems.

Polymers may be analyzed using a single molecule analysis system. A single molecule analysis system is capable of analyzing single polymers separately from other polymers. Such a system may be capable of analyzing single polymers either in a linear manner (i.e., starting at a point and then moving progressively in one direction or another) and/or in their totality. In certain embodiments in which detection is based predominately on the presence or absence of a signal such as a coincident signal, linear analysis may not be required. A linear polymer detection system is a system that analyzes polymers in a sequential or linear manner (i.e., starting at one location on the polymer and then proceeding linearly in either direction therefrom).

When detected simultaneously, the signals usually form an image of the polymer, from which distances between labels can be determined. When detected sequentially, the signals are viewed in histogram (signal intensity vs. time), that can then be translated into a profile, with knowledge of the velocity of the polymer. It is to be understood that in some embodiments, the polymer is attached to a solid support, while in others it is free flowing. In either case, the velocity of the polymer as it moves past, for example, an interaction and/or detection station will aid in determining the position of the labels, relative to each other and relative to other detectable markers that may be present on the polymer.

The invention provides in some embodiments two general classes of linear analysis, namely fixed molecule and moving molecule linear analyses. Linear analysis of fixed molecules has been described in the art and includes methods of fluid-fixing linear molecules such as DNA to surfaces and using imaging or scanning-based approaches to collect sequence information. Linear analysis of moving molecules employing either flow or electrophoretic systems are described in PCT applications WO98/35012, WO00/09757 and WO01/13088, which were published on Aug. 13, 1998, Feb. 24, 2000 and Feb. 22, 2001, respectively, and U.S. Pat. No. 6,355,420 B1, issued on Mar. 12, 2002, the entire contents of which are incorporated herein in their entirety.

The polymer may be attached to a solid support at one or multiple attachment points. The nature of the solid support is not limiting to the invention. The solid support may be any surface to which the polymer can be attached without comprising its integrity. Various types of solid supports are available including microchips, beads and the like. When fixed it a solid support, the polymer is immobile. In this latter embodiment, the interrogation and/or detection station of a polymer analysis system may move relative to the polymer. In a flow conformation, the polymer is able to move in a fluid. The polymer may also be attached to a support that is itself mobile, such as for example a free flowing bead.

Other single molecule nucleic acid analytical methods can also be used in the methods of the invention. These include optical mapping (Schwartz, D. C. et al., Science 262(5130):110-114 (1993); Meng, X. et al., Nature Genet. 9(4):432-438 (1995); Jing, J. et al., Proc. Natl. Acad. Sci. USA 95(14):8046-8051 (1998); and Aston, C. et al., Trends Biotechnol. 17(7):297-302 (1999)) and fiber-fluorescence in situ hybridization (fiber-FISH) (Bensimon, A. et al., Science 265(5181):2096-2098 (1997)). In optical mapping, nucleic acids are elongated in a fluid sample and fixed in the elongated conformation in a gel or on a surface. Restriction digestions are then performed on the elongated and fixed nucleic acids. Ordered restriction maps are then generated by determining the size of the restriction fragments. In fiber-FISH, nucleic acids are elongated and fixed on a surface by molecular combing. Hybridization with fluorescently labeled probes allows determination of sequence landmarks on the nucleic acids. Both methods require fixation of elongated nucleic acids. Pulse field gel electrophoresis can also be used to analyze the labeled nucleic acids. Pulse field gel electrophoresis is described by Schwartz, D. C. et al., Cell 37(1):67-75 (1984). Other nucleic acid analysis systems are described by Otobe, K. et al., Nucleic Acids Res. 29(22):E109 (2001), Bensimon, A. et al. in U.S. Pat. No. 6,248,537, issued Jun. 19, 2001, Herrick, J. et al., Chromosome Res. 7(6):409:423 (1999), Schwartz in U.S. Pat. No. 6,150,089 issued Nov. 21, 2000 and U.S. Pat. No. 6,294,136, issued Sep. 25, 2001. Other linear polymer analysis systems can also be used, and the invention is not intended to be limited to solely those listed herein.

### REFERENCES

Bedi A, Pasricha P J, Akhtar A J, Barber J P, Bedi G C, Giardiello F M et al. Inhibition of apoptosis during development of colorectal cancer. Cancer Res 1995; 55:1811-6.
Bodis S. Siziopikou K P, Schnitt S J, Harris J R, Fisher D E. Extensive apoptosis in ductal carcinoma in situ of the breast. Cancer 1996; 77:1831-5.
Kerr J F, Wyllie Ah, Currie A R. Apoptosis: a basic biological phenomenon with wide-ranging implications in tissue kinetics. Br J Cancer 1972; 26:239-57.
Pasquinelli A E and Ruvkun G. Control of developmental timing by mircoRNAs and their targets. Ann Rev Cell Dev Biol 2002; 18:495-513.
Shanmugathasan M, Jothy S. Apoptosis, anoikis and their relevance to the pathobiology of colon cancer. Pathol Int 200; 50: 273-9.

### EQUIVALENTS

It should be understood that the preceding is merely a detailed description of certain embodiments. It therefore should be apparent to those of ordinary skill in the art that various modifications and equivalents can be made without departing from the spirit and scope of the invention, and with no more than routine experimentation. It is intended to encompass all such modifications and equivalents within the scope of the appended claims.

All references, patents and patent applications that are recited in this application are incorporated by reference herein in their entirety.

1. A method for determining presence of a target polymer in a sample comprising
contacting a sample of one or more polymers with first and second polymer-specific probes labeled with first and second detectable labels respectively, and
analyzing the sample for the presence of coincident binding of the first and second polymer-specific probes to a single polymer,
wherein the first and second polymer-specific probes bind to unique and distinct target sites separated by a known minimum length within the target polymer, the first and second detectable labels are unique and distinct, and coincident binding of the first and second polymer-specific probes indicates presence of the target polymer in the sample.

2. The method of claim 1, wherein the sample is analyzed using a single molecule detection system.

3. (canceled)

4. The method of claim 1, further comprising measuring the total number of coincident binding events in the sample as an indicator of a level of the polymer.

5. The method of claim 1, wherein coincident binding of the first and second polymer-specific probes is indicative of a mutant polymer.

6. The method of claim 1, wherein the coincident binding of the first and second polymer-specific probes is indicative of presence of an apoptosis-resistant cell.

7-11. (canceled)

12. The method of claim 1, wherein the polymer is a nucleic acid.

13. The method of claim 12, wherein the nucleic acid is a DNA or RNA.

14-15. (canceled)

16. The method of claim 1, wherein the first and second detectable labels are fluorescent molecules.

17. The method of claim 1, wherein the polymer is free-flowing.

18-19. (canceled)

20. The method of claim 1, wherein the coincident binding is detected by the direct coincident detection of the first and second detectable labels.

21. The method of claim 1, wherein the coincident binding is a proximal binding of the first detectable label that is a donor FRET fluorophore and the second detectable label that is an acceptor FRET fluorophore, and is detected by a signal from the acceptor FRET fluorophore upon laser excitation of the donor FRET fluorophore.

22-29. (canceled)

30. The method of claim 1, wherein the polymer is condensed.

31. (canceled)

32. The method of claim 1, wherein the coincident event is a color coincident event.

33. The method of claim 1, wherein the sample has a nanoliter volume.

34. The method of claim 1, wherein the polymer is present at a frequency of 1 in 1,000,000 polymers in the sample.

35. The method of claim 1, wherein the probes are comprised of DNA, RNA, PNA, LNA or a combination thereof.

36. The method of claim 1, wherein unbound probes are not removed prior to analysis.

37. The method of claim 1, wherein the first and second detectable labels are provided as molecular beacons.

38. The method of claim 1, wherein at least the first or the second detectable label is attached to a nucleic acid molecule hybridized to a universal linker attached to the first or second polymer-specific probe.

39-40. (canceled)

41. The method of claim 1, wherein coincident binding is determined by confocal detection.
